(19) United States
(12) Patent Application Publication
(10) Pub. No.: US 2009/0042224 A1
(43) Pub. Date: Feb. 12, 2009

(54) METHODS FOR DIAGNOSIS AND/OR PROGNOSIS OF OVARIAN CANCER

(75) Inventors: Ingegerd Hellstrom, Seattle, WA (US); Karl Erik Hellstrom, Seattle, WA (US); Yi Yang, Lynnwood, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: WASHINGTON, UNIVERSITY OF, Seattle, WA (US)

(21) Appl. No.: 12/134,093

(22) Filed: Jun. 5, 2008

Related U.S. Application Data
(60) Provisional application No. 60/942,102, filed on Jun. 5, 2007.

(51) Int. Cl. G01N 33/53; G01N 33/566

(52) U.S. Cl. 435/7.92; 436/501; 435/7.1

(57) ABSTRACT

In one embodiment, methods are provided for assessing the presence of mesothelin-expressing tumor cells in a human subject. In another embodiment, methods are provided for monitoring the efficacy of treatment of a human cancer patient diagnosed with a mesothelin-expressing tumor.

### CROSS-REFERENCES TO RELATED APPLICATIONS

This application claims the benefit of Provisional Application No. 60/942,102 filed Jun. 5, 2007.

### FIELD OF THE INVENTION

The present invention relates to the diagnosis and/or prognosis of subjects suffering from cancer associated with mesothelin-expressing tumors and, in particular, to methods of measuring anti-mesothelin antibodies for use as an indicator of the presence of mesothelin expressing tumors and/or the clinical status of a patient undergoing treatment for a cancer associated with one or more mesothelin-expressing tumors.

### BACKGROUND OF THE INVENTION

Ovarian carcinoma (OvC) is the second most frequent and the most lethal gynecologic malignancy in the western world. Most cases are diagnosed at an advanced stage and this is reflected by a poor prognosis with the overall five-year survival rate not exceeding 35%. Ovarian carcinoma is disproportionately deadly because symptoms are vague and non-specific. Ovarian cancers shed malignant cells into the naturally occurring fluid within the abdominal cavity. These cells then have the potential to float in this fluid and frequently implant on other abdominal (peritoneal) structures including the uterus, urinary bladder, bowel and lining of the bowel wall (omentum). These cells can begin forming new tumor growths before cancer is even suspected. More than 60% of patients presenting with this disease already have stage III or stage IV disease, when it has already spread beyond the ovaries, and more than 75% of these patients die from disease, in spite of recent improvements of chemotherapy for ovarian cancer. However, if diagnosis is made early in the disease, five-year survival rates can reach 90% to 98%.

One marker for ovarian cancer that is used in serum assays for ovarian cancer is CA125 (Bast, R. C., et al., Gynecol. Oncol. 22:115-120 (1985); Einhorn, N., et al., Obstet. Gynaecol. 67:414-416 (1986); Einhorn, et al., Obstet. Gynecol. 80:14-18 (1992); Jacobs, I. J., et al., Br. Med. J. 313:1355-1358 (1996)). However, CA125 is also elevated in several non-malignant conditions (Fung, M. F., et al., J. Obstet. Gynaecol. Can., 26:717-728 (2004); Mas, M. R., et al., Dig. Liver Dis. 32:595-597 (2000); Malkasian, G. D., et al., Am. J. Obstet. Gynecol. 159:341-346 (1988)), which can lead to a false positive result.

Mesothelin is highly expressed on the surface of pancreatic cancers, ovarian cancers, mesothelioma, lung cancers, and some other cancers. See, e.g., Scholler, et al., Proc. Natl. Acad. Sci. USA 96:11531-11536 (1999); Cao, et al., Mod. Pathol. 14 (2005); Hassan, et al., Clin. Cancer Res. 10:3937-42 (2004).

Thus, there is a need to develop more effective tools for detecting potentially curable, early stage ovarian carcinoma and other cancers that have tumors that express mesothelin. There is also a need to identify patients afflicted with ovarian cancer and other cancer cells that express mesothelin who are most likely to have tumor recurrence following therapy, and to have effective tools for detecting recurrence as early as possible.

### SUMMARY OF THE INVENTION

In accordance with the foregoing, in one aspect, a method is provided for detecting the presence of mesothelin-expressing tumor cells in a human subject comprising determining the presence or amount of anti-mesothelin antibodies in a biological sample obtained from the human subject, wherein the presence or amount of anti-mesothelin antibodies in the biological sample is indicative of the presence of mesothelin-expressing tumor cells in the human subject.

In another aspect, a method is provided for monitoring the efficacy of treatment of a human cancer patient undergoing therapeutic treatment for a mesothelin-expressing tumor. The method comprises: (a) providing a biological sample from a human patient undergoing therapeutic treatment for a cancer associated with a mesothelin-expressing tumor; (b) determining the presence or amount of anti-mesothelin antibodies in the biological sample by contacting the biological sample with a polypeptide encoded by a polynucleotide that selectively hybridizes to a sequence at least 90% identical to a sequence comprising at least 20 contiguous nucleotides of SEQ ID NO: 1; and (c) comparing the presence or amount of anti-mesothelin antibodies determined in step (b) to an antibody reference value, wherein an amount of anti-mesothelin antibody greater than the antibody reference value is indicative of a positive response to the therapeutic treatment for the cancer.

In another aspect, a kit is provided for detecting the presence of mesothelin-expressing tumor cells in a human subject comprising reagents specific for detection of the presence or amount of anti-mesothelin antibodies in a biological sample obtained from a human subject and printed instructions for comparison of the detected presence or amount of anti-mesothelin antibodies with a reference standard.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 graphically illustrates the titration of sera using an ELISA to detect anti-mesothelin antibodies from one ovarian carcinoma patient with no evidence of disease (“NED”) and one healthy donor as described in Example 1, wherein the diamond symbols represent NED patient number 208, the squares represent healthy control subject number 8, and the triangles represent the negative (BSA) control;

FIG. 2A graphically illustrates the results of an anti-mesothelin antibody ELISA assay demonstrating the concentration level of antibodies to mesothelin in sera, diluted 1:20, obtained from apparently healthy women, as described in Example 2;

FIG. 2B graphically illustrates the results of an anti-mesothelin antibody ELISA assay demonstrating the concentration level of antibodies to mesothelin in sera obtained from women with non-malignant gynecological conditions excluding pelvic inflammatory disease, as described in Example 2;

FIG. 2C graphically illustrates the results of an anti-mesothelin antibody ELISA assay demonstrating the concentration level of antibodies to mesothelin in sera obtained from women with pelvic inflammatory disease, as described in Example 2;

FIG. 3A graphically illustrates the results of an anti-mesothelin antibody ELISA assay demonstrating the concentration level of antibodies to mesothelin in sera obtained from ovarian cancer patients exhibiting no clinical evidence of disease after treatment for ovarian cancer, as described in Example 2;

FIG. 3B graphically illustrates the results of an anti-soluble mesothelin-related protein (SMRP) ELISA assay demonstrating the concentration level of circulating SMRP in sera obtained from the same panel of ovarian cancer patients shown in FIG. 3A, as described in Example 3;

FIG. 4A graphically illustrates the results of an anti-mesothelin antibody ELISA assay demonstrating the concentration level of antibodies to mesothelin in sera obtained from ovarian cancer patients exhibiting clinical evidence of disease, as described in Example 2; and

FIG. 4B graphically illustrates the results of an anti-soluble mesothelin-related protein (SMRP) ELISA assay demonstrating the concentration level of circulating SMRP in sera obtained from the same panel of ovarian cancer patients shown in FIG. 4A, as described in Example 3.

### DETAILED DESCRIPTION OF THE PREFERRED EMBODIMENT

Unless specifically defined herein, all terms used herein have the same meaning as they would to one skilled in the art of the present invention.

The terms “percent identity” or “percent identical,” as applied to polypeptide sequences, such as the mesothelin polypeptide, or a portion thereof, is defined as the percentage of amino acid residues in a candidate protein sequence that are identical with the subject protein sequence (such as the amino acid sequence set forth in SEQ ID NO:2, or a portion thereof comprising at least 10 consecutive amino acid residues) after aligning the candidate and subject sequences to achieve the maximum percent identity. For example, percentage identity between two protein sequences can be determined by pairwise comparison of the two sequences using the bl2seq interface at the Web site of the National Center for Biotechnology Information (NCBI), U.S. National Library of Medicine, 8600 Rockville Pike, Bethesda, Md. 20894, U.S.A. The bl2seq interface permits sequence alignment using the BLAST tool described by Tatiana, A., et al., “Blast 2 Sequences—A New Tool for Comparing Protein and Nucleotide Sequences,” FEMS Microbiol. Lett. 174:247-250 (1999). The following alignment parameters are used: Matrix=BLOSUM62; Gap open penalty=11; Gap extension penalty=1; Gap x_dropff=50; Expect=10.0; Word size=3; and Filter=off.

The terms “percent identity” or “percent identical,” as applied to nucleic acid molecules, is the percentage of nucleotides in a candidate nucleic acid sequence that are identical with a subject nucleic acid molecule sequence (such as the nucleic acid molecule sequence set forth in SEQ ID NO: 1, or a portion thereof comprising at least 20 consecutive nucleotides) after aligning the sequences to achieve the maximum percent identity, and not considering any nucleic acid residue substitutions as part of the sequence identity. No gaps are introduced into the candidate nucleic acid sequence in order to achieve the best alignment. Nucleic acid sequence identity can be determined in the following manner. The subject polynucleotide molecule sequence is used to search a nucleic acid sequence database, such as the Genbank database, using the program BLASTN version 2.1 (based on Altschul, et al., Nucleic Acids Research 25:3389-3402 (1997)). The program is used in the ungapped mode. Default filtering is used to remove sequence homologies due to regions of low complexity as defined in Wootton, J. C., and S. Federhen, Methods in Enzymology 266:554-571 (1996). The default parameters of BLASTN are utilized.

As used herein, the term “healthy human subject” refers to an individual who is known not to suffer from cancer, such knowledge being derived from clinical data on the individual including, but not limited to, a different cancer assay to that described herein. The healthy individual is also preferably asymptomatic with respect to the early symptoms associated with mesothelin-expressing tumors such as ovarian cancer, which include, for example, rectal pressure, abdominal bloating, and swelling.

As used herein, the term “mesothelin-expressing tumor” refers to any type of cancer cells and/or tumors that are identified as having a neoplastic condition associated with an increased expression of mesothelin as compared to normal tissues, including but not limited to, ovarian cancer, mesothelioma, pancreatic carcinoma, or lung carcinoma. See, e.g., Scholler, et al., Proc. Natl. Acad. Sci. USA 96:11531-11536 (1999).

As used herein, the term “ovarian cancer” refers to any type of ovarian cancer including, but not limited to, serous ovarian cancer, non-invasive ovarian cancer, mixed phenotype ovarian cancer, mucinous ovarian cancer, endometrioid ovarian cancer, clear cell ovarian cancer, papillary serous ovarian cancer, Brenner cell, and undifferentiated adenocarcinoma.

As used herein, the term “recurrence of a tumor expressing mesothelin” refers to clinical evidence of cancer related to cells expressing mesothelin, for example, ovarian cancer, mesothelioma, pancreatic carcinoma, or lung carcinoma, or tumor cells derived therefrom based upon clinical data on the individual including, but not limited to, a different cancer assay to that described herein.

As used herein, the term “good prognosis” in the context of cancer associated with one or more mesothelin-expressing tumors (e.g., ovarian cancer) refers to patients who are likely to be cured from their disease, or to have at least a five-year tumor-free survival period following the initial diagnosis.

As used herein, the term “poor prognosis” in the context of cancer associated with one or more mesothelin-expressing tumors (e.g., ovarian cancer) refers to patients who are likely to die from their disease within a five-year period following the initial diagnosis.

In one aspect, a method is provided for detecting the presence of mesothelin-expressing tumor cells in a human subject. The method comprises determining the presence or amount of anti-mesothelin antibodies in a biological sample obtained from the human subject, wherein the presence or amount of anti-mesothelin antibodies in the biological sample indicates the presence of mesothelin-expressing tumor cells in the human subject. In one embodiment, the presence or amount of anti-mesothelin antibodies in the biological sample is determined by contacting the biological sample with a polypeptide encoded by a polynucleotide that selectively hybridizes to a sequence at least 80% identical, or at least 90% identical to a sequence comprising at least 20 contiguous nucleotides of SEQ ID NO: 1. In one embodiment, the presence or amount of anti-mesothelin antibodies in comparison to a reference standard (e.g., a negative control) is indicative of the presence of mesothelin-expressing cells, such as tumor cells in the human subject. In another embodiment, the amount of anti-mesothelin antibodies over a predetermined threshold amount is indicative of the presence of mesothelin-expressing tumor cells in a human subject.

A wide variety of biological samples may be used in the methods of the invention, including biological fluids. Non-limiting examples of biological fluids include blood, plasma, serum, ascitic fluid, urine, saliva, tears, pleural fluid, sputum, vaginal fluid (discharge), and washings obtained during a medical procedure (e.g., pelvic or other washings obtained during biopsy, endoscopy or surgery).

The methods of this aspect of the invention may be used as a diagnostic tool to distinguish between a subject suffering from a disease associated with the expression of mesothelin and a disease or disorder not associated with the expression of mesothelin. Examples of diseases associated with the expression of mesothelin include ovarian cancer, mesothelioma, pancreatic cancer, lung carcinoma, and pelvic inflammatory disease. In some embodiments, the methods of the invention may be used as a diagnostic tool to distinguish between a subject suffering from a disease related to a mesothelin-expressing tumor and a disease or disorder unrelated to the presence of mesothelin-expressing cancer cells. In such embodiments, a biological sample is obtained from a human subject suffering from at least one symptom associated with a mesothelin-expressing tumor (e.g., ovarian cancer, mesothelioma, pancreatic cancer or lung carcinoma) and assayed for the presence or amount of anti-mesothelin antibodies, wherein the presence or amount of anti-mesothelin antibodies is indicative of the presence of mesothelin-expressing tumor cells in the subject, and the absence of anti-mesothelin antibodies is indicative of a disease or disorder unrelated to the presence of mesothelin expressing cancer cells.

In one embodiment, the method of this aspect of the invention further comprises determining if the human subject having anti-mesothelin antibodies has pelvic inflammatory disease.

In another embodiment, the methods of the invention may be used as a diagnostic tool to distinguish between a subject suffering from pelvic inflammatory disease and a subject suffering from a non-malignant (benign) gynecological condition. In one embodiment of this aspect of the invention, the presence of anti-mesothelin antibodies indicates the subject is suffering from pelvic inflammatory disease, whereas the absence of anti-mesothelin antibodies indicates the subject is suffering from a non-malignant gynecological condition.

In one embodiment of the method, a biological sample is obtained from a human subject suffering from at least one symptom associated with ovarian cancer. Symptoms associated with ovarian cancer are known to those of skill in the field of medicine. Non-limiting examples of such symptoms include abdominal swelling/bloating; abdominal/pelvic pain or pressure; gastrointestinal symptoms (e.g., gas, indigestion, nausea, or changes in bowel movements); vaginal bleeding or discharge; urinary problems (e.g., urgency, burning or spasms); fatigue; fever; back pain; difficulty breathing. In some embodiments, the methods of this aspect of the invention further comprise performing at least one additional diagnostic assay for ovarian cancer on the subject, such as, for example, detecting the presence of CA125 in a biological sample, ultrasound, CT scan, MRI scan, biopsy, aspirate, and the like.

In one embodiment of the method, the presence of anti-mesothelin antibodies in a sample obtained from a human subject suffering from ovarian cancer that does not have pelvic inflammatory disease is indicative of the presence of mesothelin-expressing tumor cells in the subject.

In another embodiment of the method, a biological sample is obtained from a human subject suffering from at least one symptom associated with mesothelioma. Symptoms associated with mesothelioma are known to those of skill in the in the field of medicine. Non-limiting examples of symptoms associated with pleural mesothelioma may include difficulty in breathing, chest pain, weight loss, fever, night sweats, cough. Non-limiting examples of symptoms associated with peritoneal mesothelioma may include swelling, pain due to accumulation of fluid in the abdominal cavity, weight loss, mass in the abdomen, bowel obstruction, blood clotting abnormalities, anemia and/or fever. In some embodiments, the methods of this aspect of the invention further comprise performing at least one additional diagnostic assay for mesothelioma on the subject, such as, for example, chest x-ray, ultrasound, CT scan, MRI scan, biopsy, aspirate, and the like.

In another embodiment of the method, a biological sample is obtained from a human subject suffering from at least one symptom associated with pancreatic cancer. Symptoms associated with pancreatic cancer are well known to those of skill in the field of medicine and include, but are not limited to weight loss, loss of appetite, discomfort or pain around the stomach area, back pain and/or jaundice. In some embodiments, the methods of this aspect of the invention further comprise performing at least one additional diagnostic assay for pancreatic cancer on the subject such as, for example, ultrasound, CT scan, MRI scan, biopsy, aspirate, and the like.

In another embodiment of the method, a biological sample is obtained from a human subject suffering from at least one symptom associated with lung carcinoma. Symptoms associated with lung carcinoma are well known to those of skill in the field of medicine and include, but are not limited to coughing, hoarseness, hemoptysis, dyspnea, noncardiac chest pain, extrathoracic pain, neurologic symptoms, weight loss, and weakness/fatigue. In some embodiments, the methods of this aspect of the invention further comprise performing at least one additional diagnostic assay for lung carcinoma on the subject, such as, for example, chest x-ray, CT scan, MRI scan, biopsy, aspirate, and the like.

In another embodiment of the method, a biological sample is obtained from a human subject suffering from at least one symptom associated with pelvic inflammatory disease (PID). As used herein, PID refers to infection and inflammation of the female reproductive organs, including the uterus, fallopian tubes, ovaries, and other reproductive organs. Symptoms associated with pelvic inflammatory disease are well known to those of skill in the field of medicine and include, but are not limited to lower abdominal pain, fever, fatigue, diarrhea or vomiting, vaginal discharge with or without an unpleasant odor, pain during sexual intercourse, painful or difficult urination, irregular menstrual bleeding, and low back pain. In another embodiment, a biological sample is obtained from a human subject who is not experiencing any symptoms of PID, but who may be at risk of developing PID. Subjects at risk of developing PID are well known to those of skill in the field of medicine and include, but are not limited to subjects with sexually transmitted diseases, such as gonorrhea and/or Chlamydia, sexually active women in their childbearing years, in particular those under age 25 years, subjects with multiple sexual partners and/or whose partners have more than one sexual partner, and subjects who douche or use an intrauterine device. In some embodiments, the methods of this aspect of the invention further comprise performing at least one additional diagnostic assay for PID on the subject, such as, for example, a pelvic exam, analysis of vaginal discharge, cervical cultures, pelvic ultrasound and pelvic laparoscopy.

In one embodiment, the method of this aspect of the invention further comprises determining the presence or amount of soluble mesothelin-related peptides (SMRP) in a biological sample obtained from the human subject. The amount of SMRP detected in the biological sample may be compared to a reference standard such as an antigen reference value, wherein detection of an increased amount of SMRP in the sample as compared to the reference standard is indicative of the presence of mesothelin-expressing tumor cells in the human subject.

In another embodiment, a method is provided for monitoring the efficacy of treatment of a human cancer patient undergoing therapeutic treatment for a mesothelin-expressing tumor. The method comprises: (a) providing a biological sample from a human patient undergoing therapeutic treatment for a cancer associated with a mesothelin-expressing tumor; (b) determining the presence or amount of anti-mesothelin antibodies in the biological sample by contacting the biological sample with a polypeptide encoded by a polynucleotide that selectively hybridizes to a sequence at least 90% identical to a sequence comprising at least 20 contiguous nucleotides of SEQ ID NO: 1; and (c) comparing the determined presence or amount of anti-mesothelin antibodies to an antibody reference value wherein an amount of anti-mesothelin antibody greater than the antibody reference value is indicative of a positive response to the therapeutic treatment for the cancer.

In another embodiment, a method is provided for determining the likelihood of recurrence of a mesothelin-expressing tumor in a human patient undergoing therapeutic treatment for a cancer associated with a mesothelin-expressing tumor. The method comprises: (a) providing a biological sample from a human patient undergoing therapeutic treatment for a cancer associated with a mesothelin-expressing tumor; (b) determining the presence or amount of anti-mesothelin antibodies in the biological sample by contacting the biological sample with a polypeptide encoded by a polynucleotide that selectively hybridizes to a sequence at least 80%, such as at least 90% identical to a sequence comprising at least 20 contiguous nucleotides of SEQ ID NO:1; and (c) comparing the presence or amount of anti-mesothelin antibodies determined in step (b) to an antibody reference value, wherein an amount of anti-mesothelin antibody greater than the antibody reference value is indicative of a lower risk of mesothelin-expressing tumor recurrence and wherein an amount of anti-mesothelin antibody lower than the reference value is indicative of greater risk of mesothelin-expressing tumor recurrence in the human patient.

In accordance with various embodiments of the methods of the invention, the present inventors have generated a reproducible assay for detecting antibodies to native mesothelin (SEQ ID NO:2) and applied it to discriminate between women with clinical evidence of ovarian cancer, referred to as “alive with disease” or “AWD,” women with no clinical evidence of disease following therapy for ovarian cancer, referred to as “no clinical evidence of disease” or “NED,” and healthy women. As described in more detail herein, the methods of the invention that include the detection of antibodies to native mesothelin may be used, and optionally combined with an assay to detect SMRP, in order to detect the presence of mesothelin-expressing tumor cells, to determine the presence or likelihood of recurrence of a cancer associated with a mesothelin-expressing tumor, such as ovarian cancer, to assess the clinical status and/or prognosis of a patient suffering from a cancer associated with mesothelin-expressing tumors, and/or to monitor the efficacy of treatment of cancer in a patient.

As used herein, the term “mesothelin” protein refers to native human mesothelin, such as is isolated from body fluids from patients with ovarian carcinoma (e.g., ascites, pleural fluid, or urine), or isolated from cultured cells making mesothelin (e.g., cultured mesothelium or ovarian carcinoma cells), or made by recombinant DNA technology (e.g., in eukaryotic expression systems (e.g., COS cells)), in yeast, insert, or in bacterial expression systems. Mesothelin is a 40 kDa glycoprotein that is publicly available in the GenBank database under the accession number AAV87530 set forth as SEQ ID NO:2, which is encoded by the cDNA sequence set forth as SEQ ID NO: 1 (Genbank accession number AY743922.1), and mammalian homologs or a fragment thereof comprising at least ten consecutive residues of the protein (SEQ ID NO:2), or at least 20 consecutive nucleotides of the cDNA (SEQ ID NO:1).

It has been determined that there are at least three mesothelin variants, variant 1 (SEQ ID NO:4, encoded by SEQ ID NO:3, Genbank reference NP_{—}005814); variant 2, which has a 24-bp insert (Genbank reference NP_{—}037536), the sequence of which is hereby incorporated by reference; and variant 3, which has an 82-bp insert (Genbank reference AF180951), the sequence of which is hereby incorporated by reference. See Muminova, Z.E., et al., BMC Cancer 4:19-29 (2004); Hassan, R., et al., Clin. Cancer Res. 10:8751-3 (2004).

A recent study has shown that mesothelin variant 1, with a molecular weight of approximately 40 kDa, was found to be the form predominately expressed at the surface of cells on certain tumors and to be released into body fluids, whereas mesothelin variants 2 and 3 were found to be expressed and released less frequently. Hellstrom, I., et al., Cancer Epidemiol. Biomarkers Prev. 15(5):1014-1020 (2006).

As shown below in TABLE 1, the cDNA sequences encoding mesothelin and variants 1 and 2 are highly conserved. As shown below in TABLE 2, the mesothelin and variant 1 and variant 2 proteins are also highly conserved.

TABLE 1
Mesothelin cDNA Sequence Homology

| | | % Identity to |
|---|---|---|
| | Genbank | Mesothelin |
| Sequence Name | Reference | cDNA |
| human mesothelin (SEQ ID NO: 1) | AY743922.1 | 100% |
| human mesothelin variant 1 (SEQ ID | NM_005823.4 | 99% |
| NO: 3) | | |
| human mesothelin variant 2 | NM_013404.3 | 98% |

TABLE 2
Mesothelin Protein Sequence Homology

| | | % Identity to |
|---|---|---|
| | Genbank | Mesothelin |
| Sequence Name | Reference | Protein |
| human mesothelin (SEQ ID NO: 2) | AAV87530 | 100% |
| human mesothelin variant 1 (SEQ ID NO: 4) | NP_005814 | 99% |
| human mesothelin variant 2 | NP_037536 | 98% |

Mesothelin has been shown to be attached to the cell surface by phosphatidylinositol and is thought to have a role in cell adhesion and possibly in cell-to-cell recognition and signaling. Robinson, B., et al., The Lancet 362:1612-1616 (2003); Chang, K., et al., Cancer Res. 52:181-86 (1992). It has been shown that mesothelin can specifically bind to CA125 at the tumor cell surface and mediate heterotypic cell adhesion, suggesting that it is involved in OvC pathogenesis and progression (Rump, A., et al., J. Biol. Chem. 279:9190-9198 (2004)). This binding can be inhibited by antibodies to mesothelin. Id.

The detection of soluble mesothelin-related peptides (SMRP) has been reported to aid the diagnosis of mesothelioma (Robinson, B., et al., The Lancet 362:1612-1616 (2003)) and ovarian carcinoma (Scholler, N., et al., Proc. Natl. Acad. Sci. USA 96:11531-11536 (1999)). An ELISA assay (Scholler, et al., Proc. Natl. Acad. Sci. USA 96:11531-11536 (1999)) has been recently developed that measures circulating mesothelin variant 1 molecules (SEQ ID NO:4), (Hellstrom, et al., Cancer Epidemiol. 15:1014-1020 (2006)) in serum and other body fluids, often referred to as “SMRP.” Such an ELISA assay provides a diagnostic tool which favorably complements CA125 for the diagnosis and prognosis of ovarian carcinoma (McIntosh, et al., Gynecol. Oncology 95:9-15 (2004)), and which also may be used in the diagnosis/prognosis of mesothelioma (Robinson, et al., Lancet 362:1612-1616 (2003)). The measurement of antibodies to mesothelin that are present in a patient, in accordance with the methods of the present invention, may be used alone or in combination with the above-referenced assays to improve detection of mesothelin-expressing tumors in patients, such as patients suffering from ovarian carcinoma, mesothelioma, or other cancers.

In accordance with one embodiment of the methods of the invention, a human patient undergoing therapeutic treatment for a cancer associated with a mesothelin-expressing tumor is assessed for their clinical status and likelihood of recurrence of cancer. The methods in accordance with this embodiment may be practiced with patients previously diagnosed and treated for a mesothelin-expressing tumor, such as ovarian cancer, mesothelioma, pancreatic cancer or lung carcinoma (e.g., treated with surgery and/or previously or currently undergoing therapeutic treatment, such as chemotherapy, radiation therapy, protein therapeutics (e.g., antibodies, gene therapy, cancer vaccine therapy, stem cell transplant, or other therapy). Recurrence of ovarian cancer is a clinical recurrence as determined by the presence of one or more clinical symptoms of an ovarian cancer, such as, for example, a metastases, or alternatively, as determined in a biochemical test, immunological test, or serological test such as, for example, a cross-reactivity in a biological sample to a CA125 antibody, or other diagnostic test. Preferably, the recurrence of ovarian cancer is capable of being detected at least about 2 years from treatment, more preferably about 2-3 years from treatment, and even more preferably, about 4 or 5 or 10 years from treatment.

A 1-4 staging system is used for ovarian cancer, as described by the International Federation of Gynecology and Obstetrics (“FIGO”) staging system, which uses information obtained after surgery, which can include a total abdominal hysterectomy, removal of one or both ovaries and fallopian tubes, the omentum, and/or pelvic washings for cytology.

Stage I—limited to one or both ovaries

IA—involves one ovary; capsule intact; no tumor on ovarian surface; no malignant cells in ascites or peritoneal washings

IB—involves both ovaries; capsule intact; no tumor on ovarian surface; negative washings

IC—tumor limited to ovaries with any of the following: capsule ruptured, tumor on ovarian surface, positive washings

Stage II—pelvic extension or implants

IIA—extension or implants onto uterus or fallopian tube; negative washings

IIB—extension or implants onto other pelvic structures; negative washings

IIC—pelvic extension or implants with positive peritoneal washings

Stage III—microscopic peritoneal implants outside of the pelvis; or limited to the pelvis with extension to the small bowel or omentum

IIIA—microscopic peritoneal metastases beyond pelvis

IIIB—macroscopic peritoneal metastases beyond pelvis less than 2 cm in size

IIIC—peritoneal metastases beyond pelvis >2 cm or lymph node metastases, note: para-aortic lymph node metastases are considered regional lymph nodes

Stage IV—distant metastases—in the liver, or outside the peritoneal cavity

In accordance with some embodiments of the invention, a biological sample is obtained from a human patient (previously diagnosed with and previously treated for ovarian cancer, or currently undergoing treatment for ovarian cancer) which is assayed for the presence or concentration of anti-mesothelin antibodies. Biological samples for use in the methods of the invention include biological fluids. Non-limiting examples of biological fluids include blood, plasma, serum, ascitic fluid, urine, saliva, tears, pleural fluid, sputum, vaginal fluid (discharge) and washings obtained during a medical procedure (e.g., pelvic or other washings obtained during biopsy, endoscopy or surgery). The ability to use a sample of biological fluid to assess the clinical status of a subject with regard to a mesothelin-expressing tumor (such as ovarian cancer or other mesothelin-expressing tumors), provides relative ease as compared to obtaining a tissue biopsy sample of a tumor. Moreover, it enables monitoring of a patient during and/or post-treatment and, importantly, allows for earlier detection of recurrence and/or progression of ovarian cancer (or other mesothelin-expressing tumors).

In some embodiments of the invention, a biological sample is obtained from a human patient diagnosed with or at risk of developing pelvic inflammatory disease.

In accordance with the methods of this aspect of the invention, the concentration of anti-mesothelin antibody is measured in a biological sample obtained from a human patient. Any immunoassay may be used to measure the concentration of anti-mesothelin antibody, for example, enzyme linked immunosorbent assays (ELISA) and radioimmunoassays (RIA), western blotting, FACS analysis, and the like. More preferably, the assay will be capable of generating quantitative results. The biological sample may be diluted in a suitable buffer prior to analysis, for example, the sample may be diluted by a factor of at least 1:2, 1:5, 1:10, 1:20, 1:30, 1:40, 1:50, 1:80, 1:100, 1:200 or greater.

In one embodiment, the presence or amount of anti-mesothelin antibody in the biological sample is determined by contacting the biological sample with an SMRP polypeptide encoded by a polynucleotide that selectively hybridizes to a sequence at least 80% identical (e.g., at least 85% identical, or at least 90% identical, or at least 95% identical, or at least 99% identical) to SEQ ID NO: 1, or a fragment thereof comprising at least 20 consecutive nucleotides, (or at least 25 or 30, or at least 40, 60, or 80 consecutive nucleotides) of SEQ ID NO: 1.

In another embodiment, the presence or amount of anti-mesothelin antibody in the biological sample is determined by contacting the biological sample with an SMRP polypeptide at least 80% identical (e.g., at least 85% identical, or at least 90% identical, or at least 95% identical, or at least 99% identical) to the human soluble mesothelin-related protein provided as SEQ ID NO:2, or a fragment thereof comprising at least 10 consecutive amino acid residues, (or at least 20 or at least 30, such as at least 50 consecutive amino acid residues) of SEQ ID NO:2.

In one embodiment, the anti-mesothelin antibody presence or amount is measured in the biological sample through the use of an ELISA assay. Standard solid phase ELISA formats are particularly useful in determining the concentration of a protein or antibody from a variety of biological samples, such as serum. In one form, such an assay involves immobilizing an SMRP polypeptide or fragment thereof onto a solid matrix, such as, for example, a polystyrene or polycarbonate microwell or dipstick, a membrane, or a glass support (e.g., a glass slide). For example, an SMRP-coated well of an ELISA plate may be utilized. The biological sample is contacted with the SMRP-coated well and the anti-mesothelin antibody in the sample is bound and captured. After binding and washing to remove non-specifically bound immune complexes, the antibody-antigen complex is detected. Detection may be carried out with any suitable method, such as the addition of a second antibody linked to a label.

In accordance with various embodiments of the methods of this aspect of the invention, an anti-mesothelin antibody reference value may be obtained from a control group of apparently healthy subjects, for example, as described in Examples 1 and 2. In some embodiments, the antibody reference value is determined in an ELISA assay using serum obtained from healthy subjects diluted at least 1:20. In another embodiment, the antibody reference value is determined using serum obtained from patients with pelvic inflammatory disease. In one embodiment, the antibody reference value is determined using serum obtained from patients diagnosed with and/or previously treated for a cancer comprising mesothelin-expressing tumor cells. An exemplary ELISA assay for detecting anti-mesothelin antibody levels in blood samples is described in Example 1.

In accordance with the prognostic applications of the invention, in one embodiment the level of anti-mesothelin antibody in a biological sample obtained from an ovarian cancer patient is then compared to the antibody reference value. If the antibody concentration in the patient tested is higher than the reference value, such as at least 1.5 fold, more preferably at least two-fold or higher, with a P value of less than 0.05, and the patient has previously undergone treatment for ovarian cancer, then the patient has a reduced likelihood of recurrence of ovarian cancer. If the antibody concentration in an ovarian cancer patient is lower than the reference value, such as at least 1.5 fold or two-fold or lower, with a P value of less than 0.05, and the patient has previously undergone treatment for ovarian cancer, then the patient has an increased likelihood of recurrence of ovarian cancer. In another embodiment, the presence of anti-mesothelin antibody is determined by comparison to a negative antibody control sample and optionally also to a positive antibody control sample.

In another aspect, the invention provides a method of assessing the prognosis of a human cancer patient suffering from a mesothelin-expressing tumor. The method comprises: (a) determining the presence or amount of anti-mesothelin antibodies in a biological sample from a human patient suffering from a mesothelin-expressing tumor by contacting the biological sample with a polypeptide encoded by a polynucleotide that selectively hybridizes to a sequence that is at least 80% identical, such as at least 90% identical to a sequence comprising at least 20 contiguous nucleotides of SEQ ID NO:1; (b) determining the presence or amount of soluble mesothelin-related peptides (SMRP) encoded by a polynucleotide that selectively hybridizes to a sequence at least 80%, such as at least 90% identical to a sequence comprising at least 20 contiguous nucleotides of SEQ ID NO:1 in a biological sample from the human patient tested in step (a); and (c) comparing the amount of anti-mesothelin antibodies determined in step (a) to an antibody reference level, and comparing the amount of SMRP determined in step (b) to an antigen reference level, wherein the detection of SMRP in the sample at a lower amount than the antigen reference level, in combination with the detection of anti-mesothelin antibodies in the sample at a higher amount than the antibody reference level, is indicative of a good prognosis for the patient.

In accordance with this aspect of the invention, the method comprises the step of determining the presence and/or amount of SMRP in a biological sample obtained from a patient suffering from a mesothelin-expressing tumor, such as an ovarian cancer patient. As described above, SMRP is a soluble protein that has been found in the circulation of both healthy and cancer patients. The presence or amount of SMRP may be determined using any assay capable of detecting and/or measuring the amount of SMRP polypeptide.

In one embodiment, the concentration of an SMRP polypeptide encoded by a polynucleotide that selectively hybridizes to a sequence at least 80% identical (e.g., at least 85% identical, or at least 90% identical, or at least 95% identical, or at least 99% identical) to SEQ ID NO:1, or a fragment thereof comprising at least 20 consecutive nucleotides, (or at least 25 or 30, or at least 40, 60, or 80 consecutive nucleotides) of SEQ ID NO: 1 is measured in the biological sample.

In another embodiment, the amount of an SMRP polypeptide at least 80% identical (e.g., at least 85% identical, or at least 90% identical, or at least 95% identical, or at least 99% identical) to the human soluble mesothelin-related protein provided as SEQ ID NO:2, or a fragment thereof comprising at least 10 consecutive amino acid residues (or at least 20 or at least 30, such as at least 50 consecutive amino acid residues) of SEQ ID NO:2 is measured in the biological sample.

The concentration and/or relative amount, or detection of soluble mesothelin-related protein (SMRP) present in a biological fluid sample may be determined using any convenient method for measuring SMRP including, but not limited to, ELISA, radioimmunoassay, chemiluminescence assay, immunofluorescence staining and the like that include an antibody that specifically binds to SMRP. Other protein detection methods may also be used to measure SMRP, including mass spectroscopy, western blot, FACS, and the like. Suitable biological samples include a biological fluid selected from the group consisting of blood, plasma, serum, ascitic fluid, and urine.

Specific antibodies, including monoclonal antibodies directed against SMRP and variants thereof, can be readily prepared using conventional techniques, and may be used in such methods. Examples of suitable antibodies are shown below in TABLE 3. For example, a double determinant (“sandwich”) ELISA assay using two mAbs 569 and 4H3 (which recognize two different epitopes on the same antigen) may be used to detect SMRP in sera, as described in Scholler, N., et al., Proc. Natl. Acad. Sci. USA 96:11531-6 (1999). Other ELISA assays may be used to detect one or more variants of mesothelin using antibodies described in TABLE 3, or other antibodies against mesothelin.

TABLE 3
mAbs to Mesothelin Variants That Are Publically Available

| | Mice Immunized | | |
|---|---|---|---|
| | Against | | |
| | (All mAbs are | | |
| Name | Mouse IgG1) | Binding Specificity | Reference |
| 569 | Human ovarian | mesothelin | Scholler, N., et al., |
| | cancer cells | variants 1 and 3 | PNAS 96: 11531-6 |
| | | | (1999) |
| 4H3 | Antigen purified | mesothelin | Scholler, N., et al., |
| | from ascites via | variants 1, 2, and 3 | PNAS 96: 11531-6 |
| | immunoadsorption | | (1999) |
| 1A6- | Antigen purified | mesothelin variant 1 | Hellstrom, I., et al., |
| 10 | from ascites via | | Cancer Epidemiol |
| | immunoadsorption | | Biomarkers |
| | | | Prev 15(5): 1014-1020 |
| | | | (2006) |
| 2B10 | Mesothelin variant 2 | mesothelin variant 2 | Hellstrom, I., et al., |
| | fusion protein | | Cancer Epidemiol |
| | | | Biomarkers |
| | | | Prev 15(5): 1014-1020 |
| | | | (2006) |
| 8C8 | Mesothelin variant 3 | mesothelin variant 3 | Hellstrom, I., et al., |
| | fusion protein | | Cancer Epidemiol |
| | | | Biomarkers |
| | | | Prev 15(5): 1014-1020 |
| | | | (2006) |
| 4A10 | Mesothelin variant 3 | mesothelin variant 3 | Hellstrom, I., et al., |
| | fusion protein | | Cancer Epidemiol |
| | | | Biomarkers |
| | | | Prev 15(5): 1014-1020 |
| | | | (2006) |
| 13H5 | Mesothelin variant 3 | mesothelin variant 3 | Hellstrom, I., et al., |
| | fusion protein | | Cancer Epidemiol |
| | | | Biomarkers |
| | | | Prev 15(5): 1014-1020 |
| | | | (2006) |

In accordance with various embodiments of the methods of this aspect of the invention, an SMRP antigen reference value may be obtained from a control group of apparently healthy subjects, for example, as described in Example 3. In some embodiments, the antigen reference value is determined in an ELISA assay using serum obtained from healthy subjects. For example, in a serum sample, the serum may be diluted 1:40 and measured in an ELISA assay, where a negative control obtained from a healthy subject gives an absorbance value of zero at a dilution of 1:40 and a positive control obtained from a ovarian cancer patient gives an absorbance value of >0.2 at a dilution of 1:1,280. See Hellstrom, I., et al. Cancer Epidemiol Biomarkers Prev 15(5):1014-1020 (2006). Absorbance values may be determined by any method known in the art. For example, absorbance of light at 450 nanometers, often referred to as the optical density (OD), is commonly used. In one embodiment, the antigen reference value is determined using serum obtained from patients diagnosed with and/or previously treated for a cancer comprising mesothelin-expressing tumor cells.

In another embodiment, the SMRP in a biological sample is detected using mass spectrometry. For example, the technologies of electrospray isonisation mass spectrometry, matrix-assisted laser desorption ionization time-of-flight mass spectrometry (MALDI-TOFMS), surface-enhanced laser desorption ionization time-of-flight mass spectrometry (SELDI-TOFMS), and microcapillary reverse-phase HPLC nano-electrospray tandem mass spectrometry (μLC/MS/MS), which are commonly used in proteomic methods, are capable of analyzing small molecular weight proteins, such as SMRP, present in complex biological fluids such as serum, plasma or ascites.

It has been determined that the presence of a higher amount of SMRP in biological fluid samples obtained from an ovarian cancer patient in comparison to a control antigen reference value, in combination with the finding of a lower amount, or absence, of anti-mesothelin antibodies in a biological sample obtained from the patient is indicative of a high risk of recurrence of ovarian cancer, and an indicator of a poor prognosis with shorter survival rates, as described in Examples 2-3. Conversely, it has also been determined that the presence of a lower amount of SMRP in a biological fluid sample obtained from an ovarian cancer patient in comparison to a control antigen reference value, in combination with a finding of a higher amount of anti-mesothelin antibodies in a biological sample obtained from the patient, as compared to a control antibody reference value, is indicative of a lower risk of recurrence of ovarian cancer, and correlates with a good prognosis and longer survival rates, as described in Examples 2-3.

In some embodiments, the methods of the invention further comprise the step of determining levels of another ovarian cancer marker, such as integrin-linked kinase (INK), CA125, TADG-12, kallikrein 10, prostasin, osteopontin, creatine kinase beta, serotransferrin, neutrophil-gelatinase associated lipocalin (NGAL), CD163, or Gc-globulin in a biological sample obtained from the subject. The second marker may be detected at the DNA, RNA or protein level using conventional methods known in the art.

In another aspect, the invention provides a method of monitoring the efficacy of treatment of a human patient diagnosed with a mesothelin-expressing tumor. The method comprises: (a) determining a first concentration of anti-mesothelin antibodies in a first biological sample taken from a human patient diagnosed with a mesothelin-expressing tumor prior to initiation of treatment for cancer; (b) determining a second concentration of anti-mesothelin antibodies in a second biological sample from the human patient taken after initiation of treatment for cancer; and (c) comparing the first and second concentrations of anti-mesothelin antibodies, wherein an increase in the second concentration of anti-mesothelin antibodies as compared to the first concentration of anti-mesothelin antibodies measured in the first biological sample indicates a positive response to the treatment for cancer.

In accordance with the method of this aspect of the invention, a first biological sample is taken from a cancer patient before initiation of treatment and a second biological sample is taken from the patient at least one time after initiation of treatment. In some embodiments, plural treated biological samples from the subject (e.g., a subject in a preclinical trial) are taken over periodic intervals of time after initiation of treatment.

As used herein, the term “treatment” refers to surgical intervention or to the administration of one or more cancer inhibitory agents for the alleviation of symptoms associated with cancer, or halt of further progression or worsening of the symptoms. For example, successful treatment may include a removal of a tumor, such as a mesothelin-expressing tumor; an alleviation of symptoms or halting the progression of the disease, as measured by a reduction in the growth rate of a tumor, a halt in the growth of a tumor, a reduction in size of the tumor; partial or complete remission of the cancer; or increased survival or clinical benefit. For example, treatment of a subject suffering from a mesothelin-expressing tumor may include one or more of the following: surgery to remove one or more tumors and/or administration of a therapeutic agent, such as chemotherapy, radiation therapy, protein therapeutics (e.g., antibodies, gene therapy, cancer vaccine therapy, stem cell transplant, or other therapy).

For example, with regard to treatment for ovarian cancer, surgery is a preferred treatment. The type of surgery depends upon how widespread the cancer is when diagnosed (the cancer stage), as well as the type and grade of cancer. The surgeon may remove one (unilateral oophorectomy) or both ovaries (bilateral oophorectomy), the fallopian tubes (salpingectomy), and the uterus (hysterectomy). For some very early tumors (stage 1, low grade or low-risk disease), only the involved ovary and fallopian tube will be removed (called a “unilateral salpingo-oophorectomy,” USO), especially in young females who wish to preserve their fertility. In advanced stages of disease, as much tumor as possible is removed (debulking surgery). In cases where this type of surgery is successful, the prognosis is improved compared to patients where large tumor masses (more than 1 cm in diameter) are left behind. Chemotherapy is typically used after surgery to treat any residual disease. Chemotherapeutic agents, such as a platinum derivative (e.g., taxane) may be administered systemically, or may be administered intra-peritoneally via direct infusion into the abdominal cavity. Other examples of therapeutic agents for use in treatment of ovarian cancer include, but are not limited to protein therapeutics (e.g., antibodies), gene therapy, cancer vaccine therapy, and stem cell transplants. The methods of this aspect of the invention may also be used to measure the efficacy of candidate therapeutic agents for treatment of ovarian cancer.

The methods of this aspect of the invention may also be used to determine the clinical status of a patient after undergoing a treatment, such as surgery to remove a tumor. In accordance with this embodiment, the level of anti-mesothelin antibody in a biological sample obtained from a cancer patient that has been treated for a mesothelin-expressing tumor is then compared to the antibody reference value. If the antibody concentration in the patient tested is higher than the reference value, such as at least 1.5 fold, more preferably at least two-fold or higher, with a P value of less than 0.05, then the patient's clinical status was improved with the treatment (i.e. the patient has a reduced likelihood of recurrence of ovarian cancer). If the antibody concentration in the treated cancer patient is lower than the reference value, such as at least 1.5 fold or two-fold or lower, with a P value of less than 0.05, then the patient's clinical status was not improved with the treatment (i.e. the patient has an increased likelihood of recurrence of ovarian cancer).

In another aspect, a kit is provided for detecting the presence of mesothelin-expressing tumor cells in a human subject. The kit comprises reagents specific for detection of anti-mesothelin antibodies in a biological sample obtained from a human subject and printed instructions for comparison of the detected presence or amount of anti-mesothelin antibodies with a reference standard. The methods for detection of anti-mesothelin antibodies described herein may be performed using the kits of the invention. In one embodiment, the kit comprises a detection reagent for detecting anti-mesothelin antibodies comprising a polypeptide encoded by a polynucleotide that selectively hybridizes to a sequence that is at least 80% identical to a sequence comprising at least 20 contiguous nucleotides of SEQ ID NO: 1.

In some embodiments, the kit further comprises a reference standard selected from the group consisting of a specific numerical threshold; a negative control sample for concurrent evaluation, or statistical information correlating the amount of anti-mesothelin antibodies detected with the likelihood of the presence of mesothelin-expressing cancer cells in the subject. In some embodiments, the reference standard is a negative control sample, and wherein the negative control sample is included in the kit.

In preferred embodiments, the methods and kits of the invention are capable of use at a point-of-care location, such as a medical clinic (e.g., doctor's office), or hospital, in order to rapidly obtain test results. Point-of-care testing (POCT) refers to any hospital or medical clinic (doctor's office) employee performing any type of laboratory test outside of the central laboratory. POCT has revolutionized the continuum of patient care process by providing laboratory results efficiently at the patient's bedside for various tests such as HIV testing, urine dipstick, etc. For example, rapid tests to detect HIV antibodies have been developed that demonstrate sensitivities and specificities comparable to those of enzyme immunoassays without the need for sophisticated laboratory equipment and highly-trained technicians. POCT can be used with unprocessed whole blood or oral fluid specimens. See Branson, B. M., J. Lab Medicine 27(7/8):288-295 (2003). POCT assays may be in any assay format that allows for rapid testing, such as particle agglutination, immunoconcentration and immunochromatography.

For example, particle agglutination POCT assays for detecting anti-mesothelin antibodies may be carried out by mixing a patient specimen containing anti-mesothelin antibodies with latex particles coated with mesothelin polypeptide (antigen), and if anti-mesothelin antibody is present, cross-linking occurs within 10 to 60 minutes and results in agglutination, with results interpreted visually.

In another example of a POCT assay format for detecting anti-mesothelin antibodies, an immunoconcentration device (flow through) may be used which employs solid-phase capture technology, which involves the immobilization of mesothelin polypeptides (antigen) on a porous membrane. The patient specimen flows through the membrane and is absorbed into an absorbent pad. If anti-mesothelin antibodies are present in the specimen a dot or a line visibly forms on the membrane when developed with a signal reagent (e.g., a colloidal gold or selenium conjugate). A procedural control may also be included on the membrane.

In yet another example of a POCT assay format to detect anti-mesothelin antibodies, immunochromatographic (lateral flow) strips may be used that incorporate both antigen (mesothelin) and signal reagent into a nitrocellulose strip. The patient specimen is applied to an absorbent pad, or the specimen may be diluted in a vial of buffer into which the test device is inserted. The specimen migrates through the strip and combines with the signal reagent. A positive reaction results in a visual line on the membrane where the mesothelin antigen has been applied. A procedural control line may be applied to the strip beyond the mesothelin antigen line.

The following examples merely illustrate the best mode now contemplated for practicing the invention, but should not be construed to limit the invention.

### Example 1

This Example describes the development of an ELISA assay to measure antibodies to native mesothelin.

Materials and Methods

Assay for Anti-Mesothelin Antibodies

Native mesothelin was isolated from samples of urine of patients with metastatic ovarian cancer using Sepharose 4B conjugated with monoclonal antibody mAb 569 (Scholler, N., et al., Proc. Natl. Acad. Sci. USA 96:11531-11536 (1999)). The Sepharose 4B-mAb 569 conjugate was generated as follows. The mAb 569 was dissolved in 0.1M NaHCO₃ buffer containing 0.5 M NaCl (pH 8.5). Cyanogen-bromide activated Sepharose 4B (Sigma, St. Louis, Mo.) was washed and swelled in cold 1 mM HCl for 30 minutes and then washed with 10 volumes of water followed by 0.1M NaHCO₃/0.5M NaCl buffer. Immediately thereafter, mAb 569 was added to the washed resin at a concentration of 10 mg antibody per ml resin. Following 2 hours incubation at room temperature, unbound antibody was removed by washing with NaHCO₃/NaCl buffer, and unreacted groups were blocked by incubation with 0.2M Glycine, pH 6.0, overnight at 4° C.

Urine samples obtained from women with metastatic ovarian cancer were pretested to confirm the presence of a high level of SMRP. The pH of the urine was adjusted by addition of 1 M NaHCO₃ until it was >8.0, after which the sample was filtered. Sepharose 4B that had been conjugated with mAb 569 was washed with 10 volumes of PBS and the urine sample was added, followed by washing with 10 volumes of PBS. Subsequently, native mesothelin was eluted with 0.1M Glycine-HCl pH 4.5, after which the pH was neutralized by adding 2M Tris and the preparation dialyzed against PBS.

As an alternative source of mesothelin, the mesothelioma cell line Meso, established in the laboratory of the inventors, was adapted to grow in Iscove's modified Dulbecco's medium (IMDM) without serum. Culture supernatant was collected every fifth day during 4-12 weeks of culture, and the supernatant was frozen until use. After pooling culture supernatants and adjusting pH with NaHCO₃, the supernatants were filtered and run through a Sepharose 4B column conjugated with Mab 569. After washing the column with 10 volumes of PBS, mesothelin antigen was eluted from the column with Glycine-HCl pH.2.7.

ELISA assays were performed to confirm that the material isolated as described above from either urine or culture supernatants was mesothelin (Scholler N. et al., “Soluble member(s) of the mesothelin/megakaryocyte potentiating factor family are detectable in sera from patients with ovarian carcinoma,” Proc Natl Acad Sci USA 96:11531-11536, 1999). Protein sequencing was performed to confirm that the purified material represented mesothelin, as described below.

The purified mesothelin was diluted in Carbonate-Bicarbonate buffer at 5 ug/mL and incubated overnight to coat the wells of a 96-well ELISA plate. After blocking for 2 hours with 3% bovine serum albumin (BSA), the plate was washed with PBS-1% Tween 20. Serum samples at dilutions 1:20 and 1:80 were added to each well and incubated at room temperature for 1 hour. 3% BSA was added in some wells as a negative control. After washing the plate with PBS-Tween 20, 1:1000 diluted HRP-conjugated mouse anti-human IgG antibody (Invitrogen, Carlsbad, Calif.) was added to each well and incubated for 1 hour at room temperature. After washing the plate with PBS-Tween, SureBlue™ TMB Microwell Peroxidase Substrate (KPL, Gaithersburg, Md.) was added to each well and incubated for 15 minutes at room temperature before the interaction was terminated by adding the TMB stop solution (KPL). Optical density (OD) at 450 nanometers was measured with a DynaTech MR5000 plate reader (DynaTech Laboratories Inc., Chantilly, Va.).

Validation Testing and Stability of the Anti-Mesothelin Antibody Assay

Antibody tests were carried out on serum samples obtained from three patients, two tumor-bearing (AWD), and one with no clinical evidence of disease (NED). Ten ml of venous blood was withdrawn from each participant, and serum was separated using an established protocol (Zhang, P., et al., Electrophoresis 25:1823-1828 (2004)).

To determine the reproducibility of the anti-mesothelin antibody assay, repeated tests of the same sera were performed, and the mesothelin antibody assay gave results (measured in OD) that varied less than 10% (data not shown).

To determine the longitudinal stability of the assay, serial samples of sera were harvested from the same three OvC patients within a 4-month interval, during which time there was no detectable change in the patients' clinical status. As illustrated in TABLE 4, the ODs of individual serum samples from the same patient displayed very little variation.

TABLE 4
Mesothelin Antibody Tests on Serum Samples Harvested
From the Same Patients Within a 4 Month Period. The
Patient's Clinical Status Remained the Same Over
the Course of the Study.

| Patient | | Serum Sample | |
|---|---|---|---|
| Identifier | Clinical Status | (dil 1:20) | OD |
| 310 | Tumor-bearing (AWD) | Sample 1 | 0.148 |
| 310 | Tumor bearing (AWD) | Sample 2 | 0.171 |
| 310 | Tumor-bearing (AWD) | Sample 3 | 0.023 |
| 305 | Tumor-bearing (AWD) | Sample 1 | 1.147 |
| 305 | Tumor-bearing (AWD) | Sample 2 | 1.259 |
| 305 | Tumor-bearing (AWD) | Sample 3 | 1.103 |
| 208 | No clinical evidence of | Sample 1 | 1.110 |
| | disease after treatment (NED) | | |
| 208 | NED | Sample 2 | 1.079 |
| 208 | NED | Sample 3 | 1.062 |

As shown by the results in TABLE 4, the ELISA assay may be used to reproducibly measure antibodies to native mesothelin.

The data was further evaluated at several cut-offs for the OD (0.2, 0.5 or 1.0) and at different dilutions of serum. Unless otherwise indicated, sera tested for antibodies were diluted 1:20, and an OD of 0.5 was used as the cut-off for positive serum. All tests were performed on coded samples. Data were statistically evaluated using the Student's t test and chi square assays.

Antibody levels were measured in the same sera against mesothelin that had been purified from either urine or culture supernatants, with the same source of antigen being used in each experiment with various sources of sera. There were no significant differences between the ODs obtained when the same sera were tested against antigens from urine or supernatant (data not shown). The data was not expressed as quantitative protein units but as ODs, as done in studies by others (Cramer D W, et al. “Conditions associated with antibodies against the tumor-associated antigen MUC1 and their relationship to risk for ovarian cancer.” Cancer Epidemiol Biomarkers Prev 2005; 14: 1125-31; Ho MH, et al. “Humoral immune response to mesothelin in mesothelioma and ovarian cancer patients.” Clin Cancer Res 2005; 11: 3814-20.)

Characterization of Mesothelin Isolated from Urine or Culture Medium

Samples were purified by immunoaffinity chromatography using Mab 569 (Scholler N. et al., 1999). The purified material was sequenced to confirm that the purified material represented mesothelin. Sequence analysis was performed at the Harvard Microchemistry and Proteomics Analysis Facility by microcapillary reverse-phase high-performance liquid chromatography (HPLC) nano-electrospray tandem mass spectrometry (μLC/MS/MS) on a Thermo LTQ-Orbitrap mass spectrometer. Tandem mass spectrometry spectra were correlated with known sequences using the algorithm Sequest developed at the University of Washington (Eng, K, et al., “An approach to correlate tandem mass spectral data of peptides with amino acid sequences in a protein database,” J. Am. Soc. Mass Spectrom 5:976-989, 1994) and programs developed by Chittum et al, (“Rabbit betaglobin is extended beyond its UGA stop codon by multiple suppressions and translational reading gaps,” Biochemistry 37:10866-10870, 1998). Tandem mass spectrometry peptide sequences were reviewed for consensus with known proteins and the results manually confirmed for fidelity.

Peptides recovered from liquid chromatography-mass spectrometry were examined for unique features to identify the different mesothelin isoforms. Variant 2 contains an 8 amino acid insertion as published previously (Hellstrom, I. et al., 2006). Variants 1 and 3 contain an Asp to Asn amino acid change and additional C-terminal sequence extensions, respectively. Both antigen sources were found to contain all three mesothelin variants based on the presence of the Asp-Asn replacement (data not shown). However, a lack of peptide resolution at the C-terminal end, which was also observed previously (Hellstrom, I. et al., 2006), remained a problem for detailed sequence analysis of variants 1 and 3. It should be noted, that published sequence data may not be entirely reliable, and it is conceivable that the Asp-Asn replacement does not represent an actual protein peptide difference but rather an error in the initial sequence data retrieved from data banks and sequencing projects.

Pilot Experiments Titrating Sera from NED Patients and Healthy Controls

The amount of anti-mesothelin antibodies in sera from OvC patients with NED following therapy as well as from healthy control women was titrated. One such experiment is presented in FIG. 1. While the serum from the healthy subject #8 (square symbols) gave no higher OD at any dilution than the negative control (BSA) (triangles), the OD from patient #208 (diamonds) was higher than that of BSA at dilution 1:640, and repeat tests of the same sera gave ODs that varied with <10%. In other cases, sera from healthy controls were as reactive at dilution 1:20 as were sera from NED patient #208 and gave as high titers (data not shown). To conserve the amount of available, purified antigen, subsequent tests for antibodies (except when otherwise stated) used sera diluted 1:20 and an OD of 0.5 as the cut-off for positive serum.

In summary, this Example describes the successful development of an ELISA assay that is useful to measure antibodies to native mesothelin in serum. Further, this Example shows that the ELISA assay is reproducible and shows longitudinal stability.

### Example 2

This Example describes a retrospective study of samples obtained from ovarian cancer patients using the anti-mesothelin antibody to compare antibody levels in serum from healthy women, women with benign gynecological conditions, women with pelvic inflammatory disease (PID), ovarian cancer patients with no evidence of disease after treatment (NED), and ovarian cancer patients with clinical evidence of disease (AWD).

Patients

A retrospective study was done with serum obtained from 35 ovarian cancer patients, all Jewish Israeli women that were diagnosed and treated for OvC at the Gynecology-Oncology Department, Sheba Medical Center, from Jan. 1, 2000, to Jan. 31, 2003. All patients were routinely examined at the outpatient clinic of the Sheba Medical Center, and sera were harvested over a 12-month period beginning Feb. 1, 2003, with >75% of the patients providing at least 3 serial samples. The final evaluation of the patients' health status was carried out in February 2005. The diagnosis of OvC was confirmed by histopathology in all patients. The clinical details were extracted from the medical records and, when needed, via a telephone interview with the patient. Patients were followed every 2-3 months for the first year after completion of first line chemotherapy and every 3-4 months over the subsequent 2-4 years. All patients were treated with 6-8 cycles of standard platinum and taxane-based regimens. The study was approved by the institutional review board (IRB), using criteria similar to those in the United States, and each patient signed a written consent form. For follow up, blood was withdrawn at the time of visit, as part of a routine management scheme.

The status of the 35 cancer patients was defined as no evidence of disease (NED; n=11) or alive with disease (AWD, n=21), 14 of which died of disease during the observation period. Sera were also harvested from 34 age-matched control women who had been in- or out-patients for diseases other than cancer (benign diseases of the ovary), including 9 women who were diagnosed with pelvic inflammatory disease (PID), 14 women with endometriosis, and 7 women with ovarian cysts. In addition, sera were tested from an age-matched control group of 23 U.S. women who had no known diseases and specifically no gynecological symptoms.

All 35 OvC patients were Jewish Israeli women, as were the 34 age-matched controls with benign gynecological disease, except for 10 of the 14 women with endometriosis who were patients at the University of Washington. Age at diagnosis was 56+/−13 years (range 28-84 years). Twenty-six of the cancer patients had serous OvC, 3 had adenocarcinoma, 4 had endometrioid type carcinoma, and 2 had mucinous OvC. At the time of diagnosis, 2 patients were stage I, 1 patient was stage II, 30 were stage III, and two patients had metastatic stage IV disease.

Comparison of Anti-Mesothelin Antibodies in Sera From Study Participants. The mesothelin antibody assay described in Example 1 was applied to test one serum sample from each of 23 healthy women, one serum sample from each woman with a benign disease of the ovary (including PID), 46 serial samples from 14 OvC patients with NED, 77 serial samples from 21 OvC patients who had clinical evidence of disease (AWD). The combined data for each category was as follows:

TABLE 5
Anti-Mesothelin Antibody Assay (Serum Diluted 1:20)

| Subject Category | Number of Samples | Mean OD +/− S.D. |
|---|---|---|
| Healthy women | 23 | 0.47 +/− 0.40 |
| (n = 23) | | |
| Women with benign | 34 | 0.305 +/− 0.26 |
| disease (excluding | | |
| PID) (n = 34) | | |
| Women with PID | 9 | 0.785 +/− 0.55 |
| (n = 9) | | |
| Women with NED | 46 | 1.08 +/− 0.63 |
| (n = 14) | | |
| Women AWD (n = 21) | 77 | 0.54 +/− 0.46 |

Comparing the combined data from all tested sera, shown above in TABLE 5, the difference in mean OD between 23 sera from healthy women (0.47+/−0.40) and 46 sera from women with NED (1.08+/−0.63) was statistically significant (p<0.0001), as was the difference (p<0.0000009) between the 46 sera from patients with NED and 77 sera from AWD patients with clinical evidence of tumor (0.54+/−0.46). The p value for the difference in mean OD between the sera from healthy women and women AWD was <0.37. The p value for the difference in mean OD between the sera from healthy women and women with PID was <0.085. The difference in mean OD between the sera from women with benign gynecological disease and women with PID was statistically significant (p<0.001).

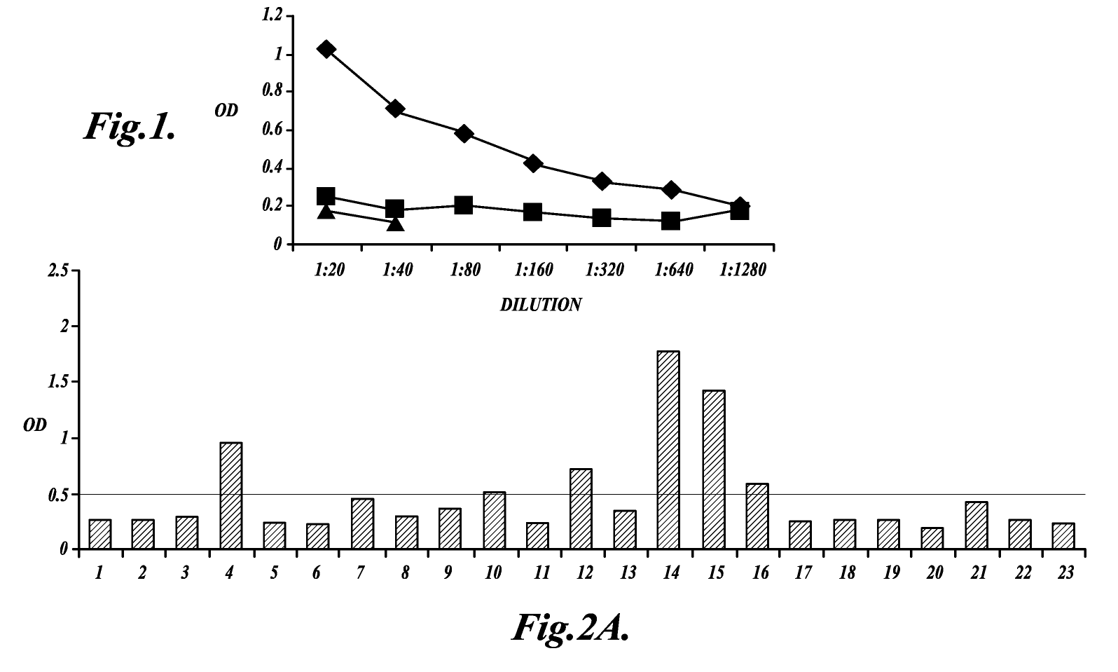

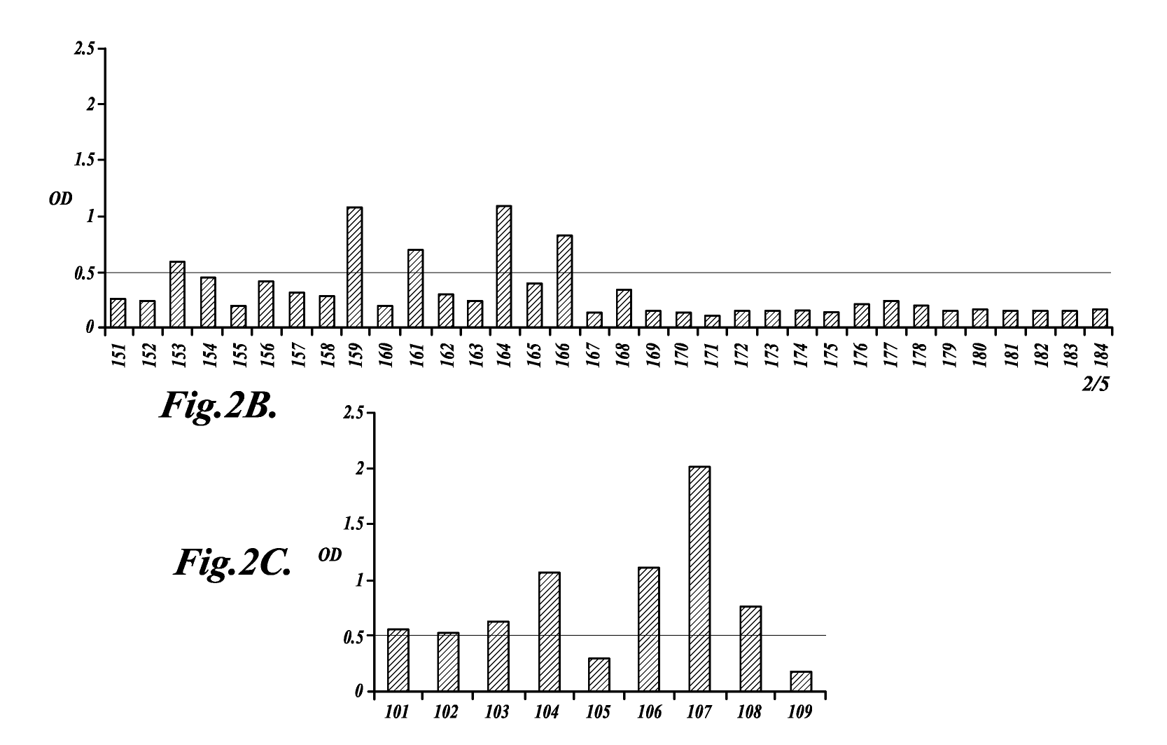

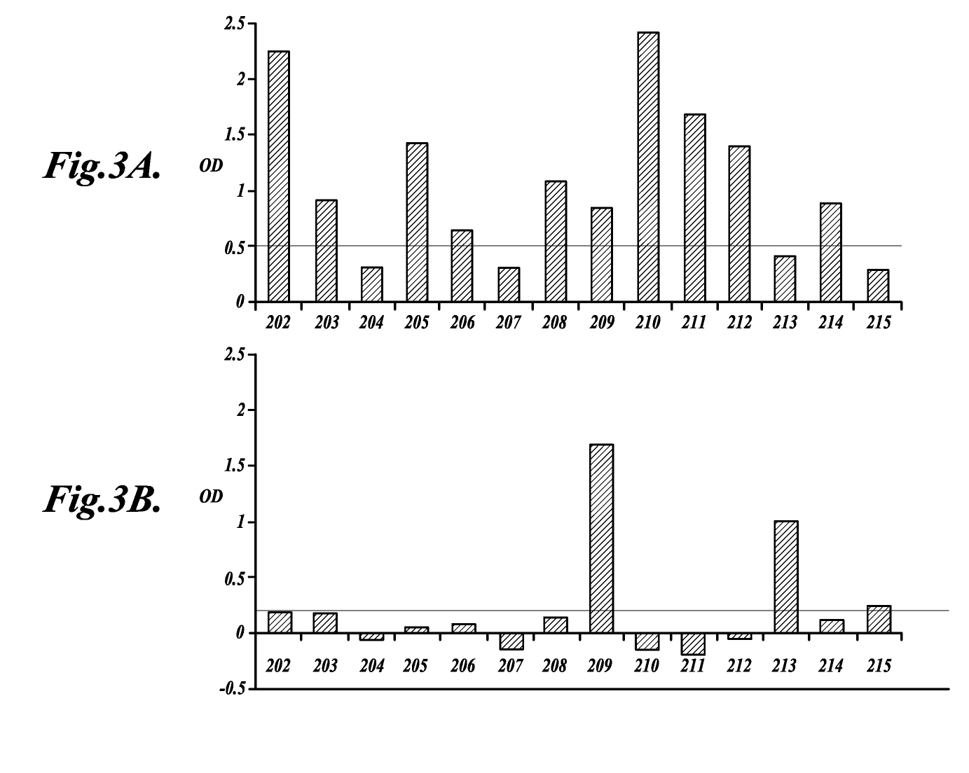

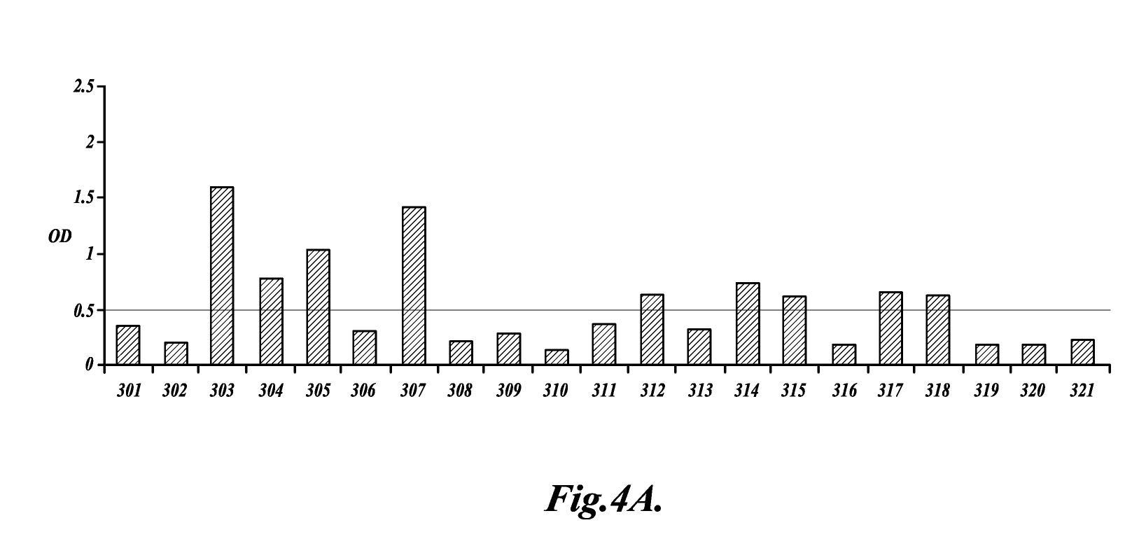

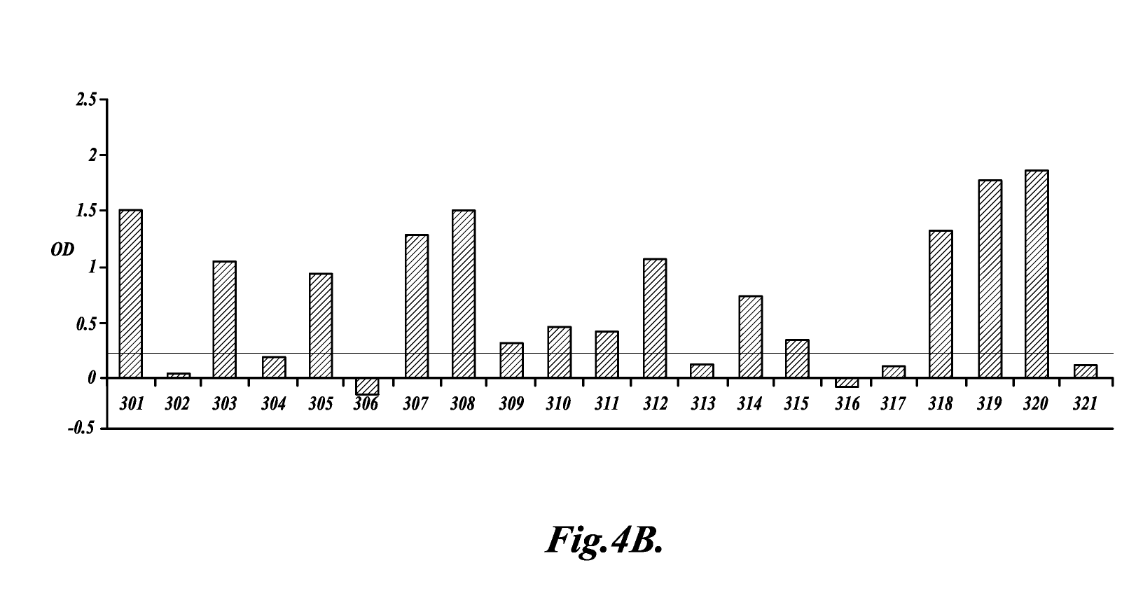

FIG. 2A shows the anti-mesothelin antibody levels in serum (diluted 1:20) obtained from 23 healthy women. FIG. 2B shows the anti-mesothelin antibody levels in serum (diluted 1:20) obtained from 34 women with non-malignant gynecological conditions, excluding PID. FIG. 2C shows the anti-mesothelin antibody levels in serum (diluted 1:20) obtained from 9 women with PID. FIG. 3A shows the anti-mesothelin antibody levels in serum (diluted 1:20) obtained from 14 ovarian cancer patients who had NED. FIG. 4A shows the anti-mesothelin antibody levels in serum (diluted 1:20) obtained from 21 ovarian cancer patients with clinical evidence of disease (AWD).

As shown in FIG. 2A, sera from 6 of the 23 healthy women (26%) had an OD>0.5, with 2 donors (9%) having an OD>1.0. The mean OD for serum diluted 1:20 was 0.474+/−0.399 (Table 5). None of the healthy women had detectable levels of circulating mesothelin antigen when tested at a dilution of 1:40 and using OD 0.2 as cut-off (data not shown).

As shown in FIG. 2B, 5 of 34 women with non-malignant gynecological conditions, but excluding PID, had sera with an OD>0.5 at a dilution of 1:20 for a mean reactivity of 0.305+/−0.262 (Table 5). Three of the 5 women whose sera were positive with the criteria applied had ovarian cysts. All 14 women with endometriosis were negative for anti-mesothelin antibodies, including 4 from Israel (#171-174) and 10 from the United States (#175-184).

Serum samples were also tested from 9 women with PID. As shown in FIG. 2C, sera from 7 (78%) of these donors had an OD>0.5, and 3 (33%) had an OD>1.0. The mean OD was 0.785+/−0.549 (Table 5). The difference in mean OD for sera from women with PID was different from that of sera from women with other benign gynecological diseases (p<0.001); the p value in comparison with sera from healthy women was <0.085.

As shown in FIG. 3A, 10 of 14 NED patients (71%) had an OD>0.5 and 6 NED patients (43%) had an OD>1.0. As shown in FIG. 4A, the anti-mesothelin antibody levels in AWD patients fell between the healthy and NED patient groups, with 9 of 21 patients (43%) having an OD>0.5 and 3 patients (14%) having an OD>1.0. These data are summarized below in TABLE 6. TABLE 7 summarizes the data from sera diluted 1:80, with a cutoff value of OD 0.5 or 0.2. Note that women with benign (non-malignant) disease were not tested at the 1:80 dilution.

TABLE 6
Relationship Between Clinical Status and Anti-Mesothelin
Antibodies in Serum Diluted 1:20, at Two Different Cut-Offs
(OD > 0.5 and OD > 1.0)

| Subject | Number of | OD > 0.5 | OD > 0.1 |
|---|---|---|---|
| Category | Subjects | (dil 1:20) | (dil 1:20) |
| Healthy | 23 | 6 (26%) | 2 (9%) |
| Women with | 34 | 5 (15%) | 2 (7%) |
| benign disease | | | |
| (excluding | | | |
| PID) | | | |
| NED | 14 | 10 (71%)*,# | 6 (43%)** |
| AWD | 21 | 9 (43%) | 3 (14%) |

*p < 0.01 in comparison with serum from healthy women
**p < 0.025 in comparison with serum from healthy women
#p < 0.001 in comparison with serum from women with benign disease excluding PID

TABLE 7
Relationship Between Clinical Status and Anti-Mesothelin
Antibodies in Serum Diluted 1:80, at Two Different Cut-Offs
(OD > 0.5 and OD > 1.0)

| Subject | Number of | OD > 0.5 | OD > 0.2 |
|---|---|---|---|
| Category | Subjects | (dil 1:80) | (dil 1:80) |
| Healthy | 23 | 2 (9%) | 7 (30%) |
| NED | 14 | 7 (50%)* | 11 (79%)* |
| AWD | 21 | 4 (19%)** | 13 (62%)*** |

*p < 0.01 in comparison with serum from healthy women
**p < 0.05 in comparison with serum from patients with NED
***p < 0.025 in comparison with serum from healthy women

As shown above in TABLE 6 and TABLE 7, the same relative differences were maintained in sera from healthy women, ovarian cancer patients with NED and patients AWD. In agreement with previously published data by Ho, et al., the results presented above show that many OvC patients make antibodies to mesothelin which, like most other tumor-associated antigens, can induce an immune response in the tumor-bearing host. Ho, et al., Clin. Cancer Res. 11:3814-3820 (2005).

However, in contrast to the results published in the Ho, et al. study, anti-mesothelin antibodies were detected in a substantial fraction of healthy individuals. It is likely that the detection of anti-mesothelin antibodies in healthy individuals was observed due to the use of a lesser dilution (1:20) of the sera in the present study as compared to the Ho, et al. study. In the present study a significant difference in anti-mesothelin antibody concentration was observed between sera from patients with OvC and healthy women, with the difference being most pronounced when testing sera from the NED group.

Seven of the 9 women diagnosed with inflammatory pelvic disease (78%) had high antibody levels, as compared to a much lower percentage of women with other benign gynecological diseases (15%) or healthy women (26%).

While not wishing to be bound by theory, the observed protective effect of anti-mesothelin antibodies with regard to the development and progression of OvC may be due to the fact that anti-mesothelin antibodies have been shown to prevent the binding of mesothelin to CA125 and thereby impact cellular adhesion, as demonstrated in vitro. Rump, A., et al., J. Biol. Chem. 279:9190-9198 (2004). Furthermore, it is known that antibodies can be cytotoxic in the presence of complement, can mediate antibody-dependent cellular cytotoxicity in the presence of NK cells or macrophages, and can also have an impact of the generation and expansion of T cell responses to tumor antigens. Hellstrom, K. E., et al., Expert Rev. Vaccines 2:517-532 (2003). Therefore, it is likely that the presence of both mesothelin antigen and anti-mesothelin antibodies will result in the formation of immune complexes of various sizes.

It is known that immune complexes can be preferentially taken up by the Fc receptors of APC, and their amounts and relative composition may determine whether this will lead to the generation/expansion of a potentially tumor-destructive Th1 type immune response or inhibit it, e.g., by stimulating the formation of suppressor/Treg cells. Gershon, R. K., et al., Nature 250:594-596 (1974). Anti-cancer therapy is likely to influence antibody formation, both by decreasing the number of tumor cells releasing antigen and by acting directly on antibody forming cells, as in the case of cytotoxic drugs. Therefore, changes in antibody levels are likely to influence the amount of SMRP that is detectable by ELISA.

### Example 3

This Example describes the assessment of serum SMRP levels in study participants described in Example 2.

Elisa for Serum SMRP Levels

Sera obtained from each participant described in Example 2 was diluted 1:40 with PBS containing 3% BSA. Serum SMRP levels were determined by a sandwich ELISA using 2 mAbs (OV569 and 4H3), which bind to different SMRP epitopes (Scholler, N., et al., Proc. Natl. Acad. Sci. USA 96:11531-11536 (1999); Hellstrom, I., et al., Cancer Epidemiol. Biomarkers Prev. 15:1014-1020 (2006)). SMRP levels were determined as optical density (OD) according to absorbance measurement by an ELISA plate reader at 450 nm (Scholler, N., et al., Proc. Natl. Acad. Sci. USA 96:11531-11536 (1999)). A serum is classified as positive for SMRP when the OD at dilution 1:40 is above the commonly accepted cut-off of 0.20 OD (Scholler, et al., Proc. Natl. Acad. Sci. USA96:11531-11536 (1999); Robinson, B., et al., Lancet362:1612-1616 (2003); McIntosh, M., et al., Gynecologic Oncology 95:9-15 (2004)), which corresponds to 3 standard deviations (SD) above the mean absorbance measurement at 460 nm as previously determined with a group of >100 healthy controls (I. Hellstrom, unpublished findings).

Results

FIG. 3B shows the SMRP peptide levels measured in serum (diluted 1:40) obtained from 14 ovarian cancer patients who had NED. FIG. 4B shows the SMRP peptide levels measured in serum (diluted 1:40) obtained from 21 ovarian cancer patients with clinical evidence of disease (AWD). As shown in FIG. 3B, in the NED group, 3 of 14 patients (21%) had sera that were positive for SMRP when tested at a dilution of 1:40. Eleven of 14 patients (79%) had ODs less than the 0.20 cut-off level. Nine of those patients (64%) tested positive for antibodies to mesothelin (as discussed in Example 2), while two patients had neither detectable antibodies to mesothelin nor detectable SMRP. One patient was positive for both anti-mesothelin antibodies and SMRP, and two patients were positive for SMRP and negative for anti-mesothelin antibodies. In contrast, as shown in FIG. 4B, sera from 15 of 21 AWD patients (71%) were positive for SMRP, a result which is significantly different from the NED group (p<0.005). Only one of 21 patients (5%) in the AWD group had anti-mesothelin antibodies and no detectable SMRP, 7 patients (33%) had SMRP and no anti-mesothelin antibodies, and 8 patients (38%) had both anti-mesothelin antibodies and SMRP. These results are summarized below in TABLE 8.

TABLE 8
Relationship Between Clinical Status, SMRP In Serum, and
Anti-Mesothelin Antibodies in Patients With OvC

| SMRP Antigen/ | Number of OvC Patients | Number of OvC Patients |
|---|---|---|
| Antibody Status | with NED | with Tumor (AWD) |
| Ag+Ab+ | 1 | 8 (p < 0.05) |
| Ag+Ab− | 2 | 7 |
| Ag−Ab+ | 9 (p < 0.0002) | 1 |
| Ag−Ab− | 2 | 5 |

These results demonstrate that the majority of ovarian cancer patients who are clinically tumor free following therapy (NED) have antibodies to native mesothelin and do not have detectable circulating SMRP. In contrast, patients with clinical evidence of tumors (AWD) have circulating SMRP and either do, or do not have antibodies to mesothelin. Therefore, the presence of antibodies in the absence of circulating antigen correlates with a low, clinically undetectable tumor load (NED).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for detecting the presence of mesothelin-expressing tumor cells in a human subject comprising determining the presence or amount of anti-mesothelin antibodies in a biological sample obtained from the human subject, wherein the presence or amount of anti-mesothelin antibodies in the biological sample is indicative of the presence of mesothelin-expressing tumor cells in the human subject.

2. The method of claim 1, wherein the presence or amount of anti-mesothelin antibodies in the biological sample is determined by contacting the biological sample with a polypeptide encoded by a polynucleotide that selectively hybridizes to a sequence at least 90% identical to a sequence comprising at least 20 contiguous nucleotides of SEQ ID NO: 1.

3. The method of claim 1, further comprising comparing the determined amount of anti-mesothelin antibodies to a reference standard, wherein an amount of anti-mesothelin antibody detected greater than the reference standard is indicative of the presence of mesothelin-expressing tumor cells in the human subject.

4. The method of claim 1, further comprising determining if the human subject with anti-mesothelin antibodies is suffering from a disease selected from the group consisting of ovarian cancer, mesothelioma, pancreatic cancer, and lung carcinoma.

5. The method of claim 1, further comprising performing at least one diagnostic assay to determine if the human subject with anti-mesothelin antibodies has pelvic inflammatory disease.

6. The method of claim 5, wherein the presence of anti-mesothelin antibodies in the sample obtained from the human subject and the absence of pelvic inflammatory disease in the subject is indicative of the presence of mesothelin-expressing tumor cells in the subject.

7. The method of claim 1 wherein the human subject is undergoing therapeutic treatment for a cancer associated with a mesothelin-expressing tumor.

8. The method of claim 1, wherein the biological sample is a biological fluid selected from the group consisting of blood, plasma, serum, ascitic fluid, urine, saliva, tears, pleural fluid, sputum, vaginal fluid, and washings obtained during a medical procedure.

9. A method of monitoring the efficacy of treatment of a human cancer patient undergoing therapeutic treatment for a mesothelin-expressing tumor, the method comprising:
(a) providing a biological sample from a human patient undergoing therapeutic treatment for a cancer associated with a mesothelin-expressing tumor;
(b) determining the presence or amount of anti-mesothelin antibodies in the biological sample by contacting the biological sample with a polypeptide encoded by a polynucleotide that selectively hybridizes to a sequence at least 90% identical to a sequence comprising at least 20 contiguous nucleotides of SEQ ID NO: 1; and
(c) comparing the determined presence or amount of anti-mesothelin antibodies to an antibody reference value wherein an amount of anti-mesothelin antibody greater than the antibody reference value is indicative of a positive response to the therapeutic treatment for the cancer.

10. The method of claim 9, wherein the antibody reference value is determined from a biological sample obtained from healthy control subjects.

11. The method of claim 9, wherein the antibody reference value is determined from a biological sample obtained from the patient prior to treatment for cancer.

12. The method of claim 9, further comprising determining if the human patient that has anti-mesothelin antibodies has pelvic inflammatory disease.

13. The method of claim 9, wherein the antibody specifically binds to a polypeptide encoded by a polynucleotide that selectively hybridizes to a sequence at least 95% identical to a sequence comprising at least 20 contiguous nucleotides of SEQ ID NO:3.

14. The method of claim 9, wherein the antibody specifically binds to a polypeptide comprising a sequence at least 95% identical to a sequence comprising at least 10 contiguous amino acids of SEQ ID NO:4.

15. The method of claim 9, wherein the biological sample is a biological fluid selected from the group consisting of blood, plasma, serum, ascitic fluid, urine, saliva, tears, pleural fluid, sputum, vaginal fluid, and washings obtained during a medical procedure.

16. The method of claim 9, wherein the biological sample is serum.

17. The method of claim 9, wherein the amount of anti-mesothelin antibodies in the serum is determined using an ELISA assay.

18. The method of claim 9, further comprising the step of determining the presence or amount of soluble mesothelin-related peptides (SMRP) encoded by a polynucleotide that selectively hybridizes to a sequence at least 90% identical to a sequence comprising at least 20 contiguous nucleotides of SEQ ID NO: 1 in the biological sample, and comparing the determined amount of the polypeptide to an antigen reference value, wherein the detection of a lower amount of the polypeptide in the sample as compared to the antigen reference value in combination with the detection of an increased amount of anti-mesothelin antibodies in the sample, as compared to the antibody reference value, is indicative of a positive response to the treatment for cancer.

19. The method of claim 18, wherein the antigen reference value is determined from a biological sample obtained from healthy control subjects.

20. The method of claim 18, wherein the antigen reference value is determined from a biological sample obtained from the human patient prior to treatment for cancer.

21. The method of claim 9, wherein the human patient is suffering from a disease selected from the group consisting of ovarian cancer, mesothelioma, pancreatic carcinoma, and lung carcinoma.

22. The method of claim 9, wherein the treatment includes administration of at least one of a chemotherapeutic agent, radiation treatment, antibody therapy, cancer vaccine therapy, gene therapy, or stem cell transplant.

23. The method of claim 9, wherein the treatment includes surgery to remove at least a portion of a tumor-expressing mesothelin.

24. A kit for detecting the presence of mesothelin-expressing tumor cells in a human subject, the kit comprising reagents specific for detection of the presence or amount of anti-mesothelin antibodies in a biological sample obtained from a human subject and printed instructions for comparison of the detected presence or amount of anti-mesothelin antibodies with a reference standard.

25. The kit of claim 24, wherein the reference standard is selected from the group consisting of a specific numerical threshold, a negative control sample for concurrent evaluation, or statistical information correlating the amount of anti-mesothelin antibodies detected with the likelihood of the presence of mesothelin-expressing tumor cells in the subject.

26. The kit of claim 24, wherein the reference standard is a negative control sample, and wherein the negative control sample is included in the kit.

27. The kit of claim 26, wherein the reagents specific for detection of anti-mesothelin antibodies comprise a polypeptide encoded by a polynucleotide that selectively hybridizes to a sequence that is at least 80% identical to a sequence comprising at least 20 contiguous nucleotides of SEQ ID NO: 1.

28. The kit of claim 24, further comprising at least one reagent for detecting the presence of pelvic inflammatory disease in the human subject.


SEQUENCE LISTING (4 sequences)

SEQ ID NO: 1 - DNA, 2187 bp, Homo sapien
  CDS (169)..(2037)
tgccaggctctccacccccacttcccaattgaggaaaccgaggcagaggaggctcagcgccacgcactcctctttctgcctggccggccactcccgtctgctgtgacgcgcggacagagagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccatttcttctcccgcatcacgaaagccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgcttagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccctgttccaccccaagagaactcgcgctcagtaaacgggaacatgccccctgcaaaaaaaaaaaaaaaaaaaaaaaaaa

SEQ ID NO: 2 - protein, 622 aa, Homo sapien [sequence omitted]

SEQ ID NO: 3 - DNA, 2052 bp, Homo sapien
  CDS (52)..(1920)
agagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccccgttccaccccaagagaactcgcgctcagtaaacgggaacatgccccctgcagacacgt

SEQ ID NO: 4 - protein, 622 aa, Homo sapien
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA